(19) United States
(12) Patent Application Publication
(10) Pub. No.: US 2020/0315435 A1
(43) Pub. Date: Oct. 8, 2020

(54) NOVEL 360-DEGREE PANORAMIC VIEW FORMED FOR ENDOSCOPE ADAPTED THERETO WITH MULTIPLE CAMERAS, AND APPLICATIONS THEREOF TO REDUCE POLYP MISS RATE AND FACILITATE TARGETED POLYP REMOVAL

(71) Applicant: Nitesh Ratnakar, Wheeling, WV (US)

(72) Inventor: Nitesh Ratnakar, Wheeling, WV (US)

(21) Appl. No.: 16/786,979

(22) Filed: Feb. 10, 2020

Related U.S. Application Data
(63) Continuation of application No. 15/263,360, filed on Sep. 12, 2016, now abandoned.
(60) Provisional application No. 62/217,015, filed on Sep. 10, 2015.
(60) Provisional application No. 62/221,107, filed on Sep. 21, 2015.

(51) Int. Cl. A61B 1/00; A61B 1/05

(52) U.S. Cl. CPC A61B 1/00181; A61B 1/00009; A61B 1/051; A61B 1/00177; A61B 1/0005

(57) ABSTRACT

Disclosed is an endoscope system comprising an endoscope having at its distal end at least one front-facing camera and multiple side-facing cameras, with each of the front-facing and side-facing cameras capturing a corresponding participating sub-view. The endoscope system further comprises an image display device adapted to receive the corresponding participating sub-views, and integrate and combine the corresponding participating sub-views to form a combination panoramic view covering or substantially covering a vicinity of 360-degree or substantially 360-degree surrounding the distal end.

### CROSS-REFERENCE TO RELATED APPLICATION

This application is a continuation application claiming priority under 35 U.S.C. § 120 to currently pending U.S. Non-provisional patent application Ser. No. 15/263,360, filed on Sep. 12, 2016, which is an application claiming the benefit under 35 U.S.C. § 119(e) of Provisional Patent Application Nos. 62/217,015 and 62/221,107, respectively filed Sep. 10, 2015 and Sep. 21, 2015, both related to U.S. Non-provisional patent application Ser. No. 14/736,258, filed on Jun. 10, 2015 and titled “Endoscope With Multiple Views And Novel Configurations Adapted Thereto.” The entire disclosures of all of said prior applications are hereby incorporated by reference.

### RELATED ART

FIGS. 1A-C illustrate limitations of a conventional endoscope with only a single front view camera (which may also be referred to as “front camera” or “front-facing camera”). As shown in FIGS. 1A-C, when, e.g., deployed in a colon of a human, the shown conventional endoscope, even with a maximum 170-degree field of view (FOV), still misses a polyp “hidden” behind one or more colon folds, as the one or more colon folds obstruct the capturing of an image of the “hidden” polyp and thus render the “hidden” polyp not visible in the FOV of the front view camera, which is a less desirable situation that needs to be improved or corrected.

### BRIEF SUMMARY

In one aspect, the present disclosure provides an endoscope system comprising an endoscope having at its distal end at least one front-facing camera and multiple side-facing cameras, with each of the front-facing and side-facing cameras capturing a corresponding participating sub-view. The endoscope system further comprises an image display device adapted to receive the corresponding participating sub-views, and integrate and combine the corresponding participating sub-views to form a combination panoramic view covering or substantially covering a vicinity of 360-degree or substantially 360-degree surrounding the distal end.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures, unless expressly specified, have not necessarily been drawn to scale. Also, any text and/or any numerical data (numbers) appeared on any drawing figures is provided to illustrate an exemplary embodiment or implementation, and thus is provided for the purpose of illustration and not for the purpose of limitation. For example, the dimensions of some of the elements may be exaggerated relative to other elements. Embodiments incorporating teachings of the present disclosure are shown and described with respect to the figures presented herein, in which:

FIGS. 1A-C illustrate limitations of a conventional endoscope with only a single front view camera (which may also be referred to as “front camera” or “front-facing camera”).

FIGS. 2A-C illustrate a novel 360-degree panoramic view as well as exemplary configurations of the disclosed endoscope adapted to realize same, according to one or more embodiments of the present disclosure.

FIGS. 3A-D illustrate how the novel 360-degree panoramic view may be used to locate a polyp “hidden” from the center sub-view (a polyp which would have been missed by a conventional front-view-only endoscope, and may even likely be missed by a prior art multi-view endoscope not capable of forming, or otherwise not provided with, the novel 360-degree panoramic view), according to one or more embodiments of the present disclosure.

FIGS. 4A-C illustrate channels through which the disclosed endoscope (adapted to provide an embodiment of the novel 360-degree panoramic view) may be used to perform polyp (lesion) removal during a withdrawal of the disclosed endoscope from inside a colon, according to one or more embodiments of the present disclosure.

FIGS. 5A-B illustrate how the distal section of an embodiment of the disclosed endoscope may be flexed horizontally or vertically from a current location (position) to a new location (position), so as to advantageously change the direction which the front face of the distal end of the endoscope faces for the strategic purpose of, e.g., re-establishing a therapeutic zone in the immediate front of the front face of the distal end of the endoscope shaft, according to one or more embodiments of the present disclosure.

FIG. 6 illustrates an example of how an embodiment of the disclosed novel 360-degree panoramic view may work in concert (conjunction) with an embodiment of the disclosed endoscope (adapted to form and realize the 360-degree panoramic view) so as to effectively reduce polyp miss rate as well as target, and facilitate the removal of, a located and discovered polyp (lesion) during a withdrawal of the endoscope shaft from inside a colon, according to one or more embodiments of the present disclosure.

FIG. 7 illustrates an innovative and novel lighting scheme which may be incorporated into the disclosed endoscope to realize the novel 360-degree panoramic view, according to one or more embodiments of the present disclosure.

FIG. 8 illustrates an innovative and novel “heads up display” scheme which may be incorporated into the disclosed endoscope, according to one or more embodiments of the present disclosure.

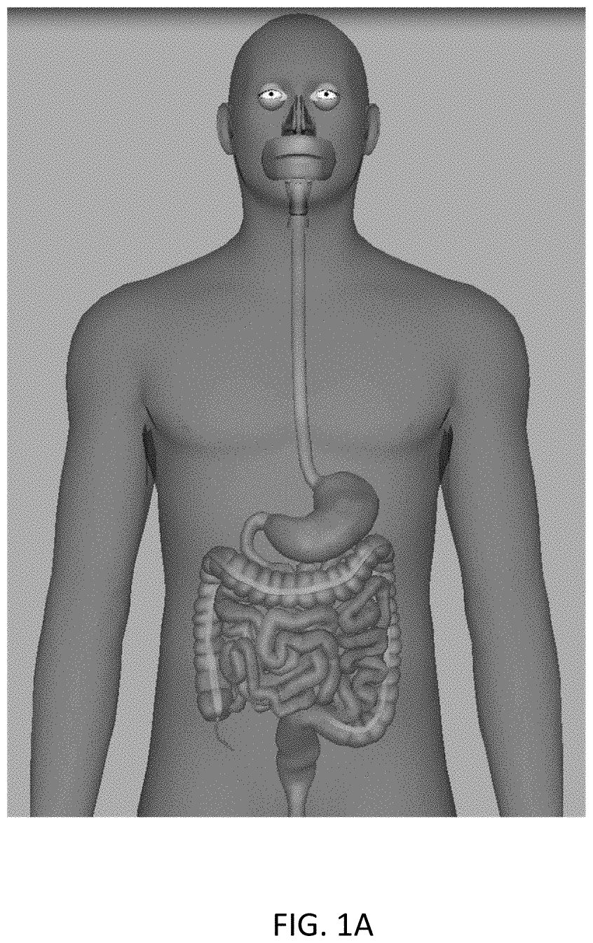

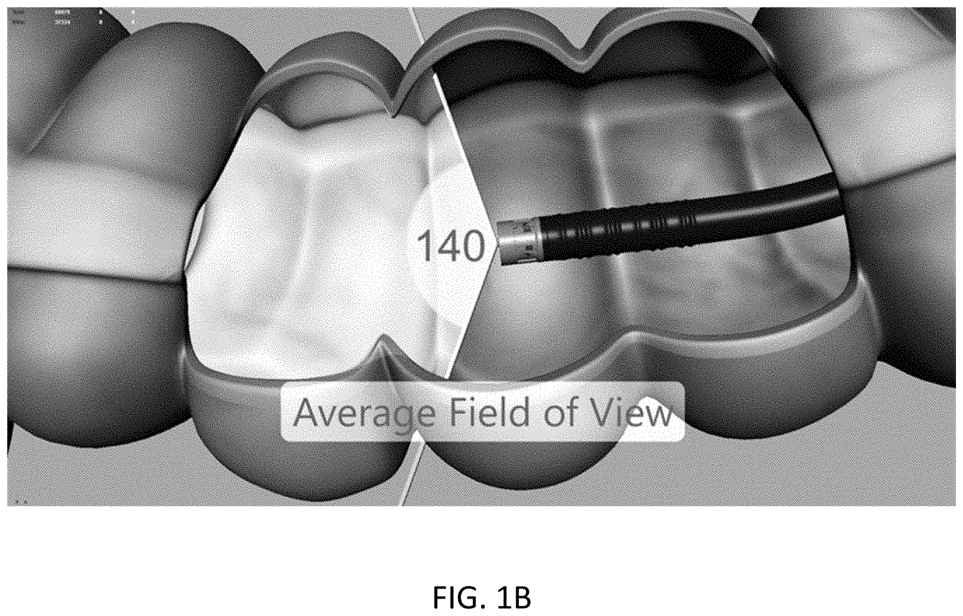

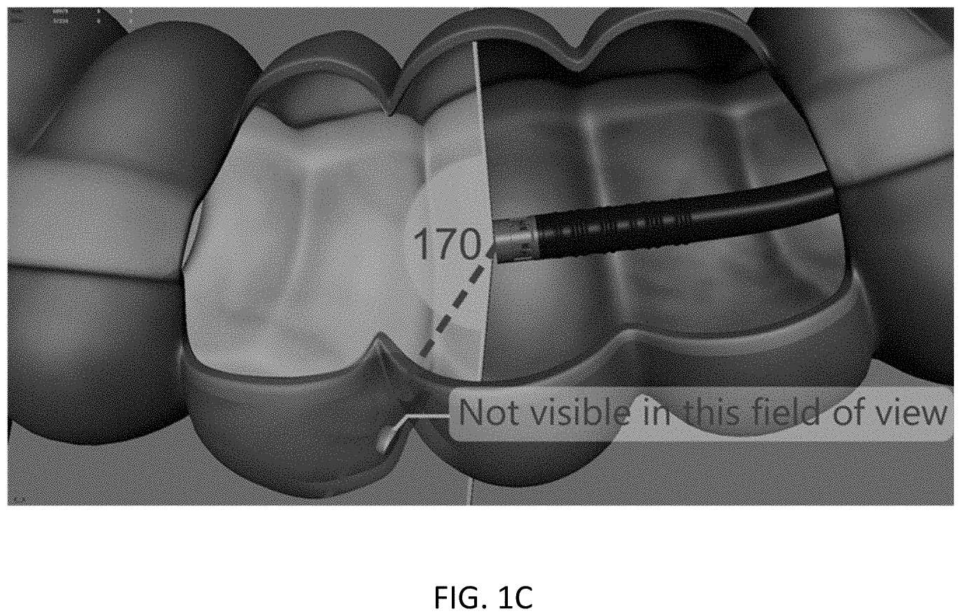

### DETAILED DESCRIPTION

In the following detailed description of exemplary embodiments of the disclosure, specific exemplary embodiments in which the disclosure may be practiced are described in sufficient detail to enable those skilled in the art to practice the disclosed embodiments. For example, specific details such as specific method orders, structures, elements, and connections have been presented herein. However, it is to be understood that the specific details presented need not be utilized to practice embodiments of the present disclosure. The following detailed description is, therefore, not to be taken in a limiting sense, and the scope of the present disclosure is defined by the appended claims and equivalents thereof.

References within the specification to “one embodiment,” “an embodiment,” “embodiments”, or “one or more embodiments” are intended to indicate that a particular feature, structure, or characteristic described in connection with the embodiment is included in at least one embodiment of the present disclosure. The appearance of such phrases in various places within the specification are not necessarily all referring to the same embodiment, nor are separate or alternative embodiments mutually exclusive of other embodiments. Further, various features are described which may be exhibited by some embodiments and not by others. Similarly, various requirements are described which may be requirements for some embodiments but not other embodiments.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. Moreover, the use of the terms first, second, etc. do not denote any order or importance, but rather the terms first, second, etc. are used to distinguish one element from another.

Those of ordinary skill in the art will appreciate that the components and basic configuration depicted in the following figures may vary. Other similar or equivalent components may be used in addition to or in place of the components depicted. A depicted example is not meant to imply limitations with respect to the presently described one or more embodiments and/or the general disclosure.

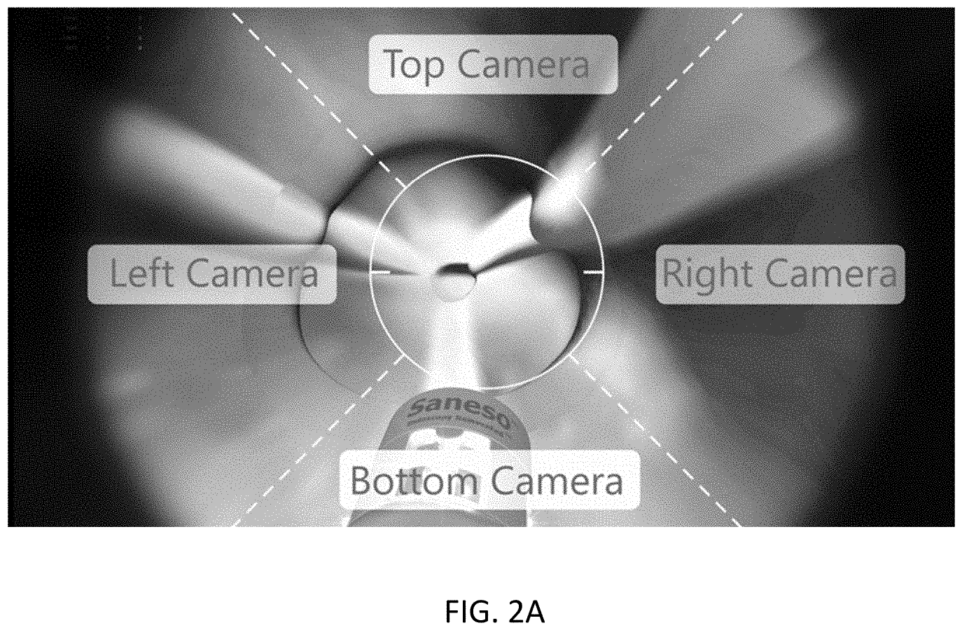

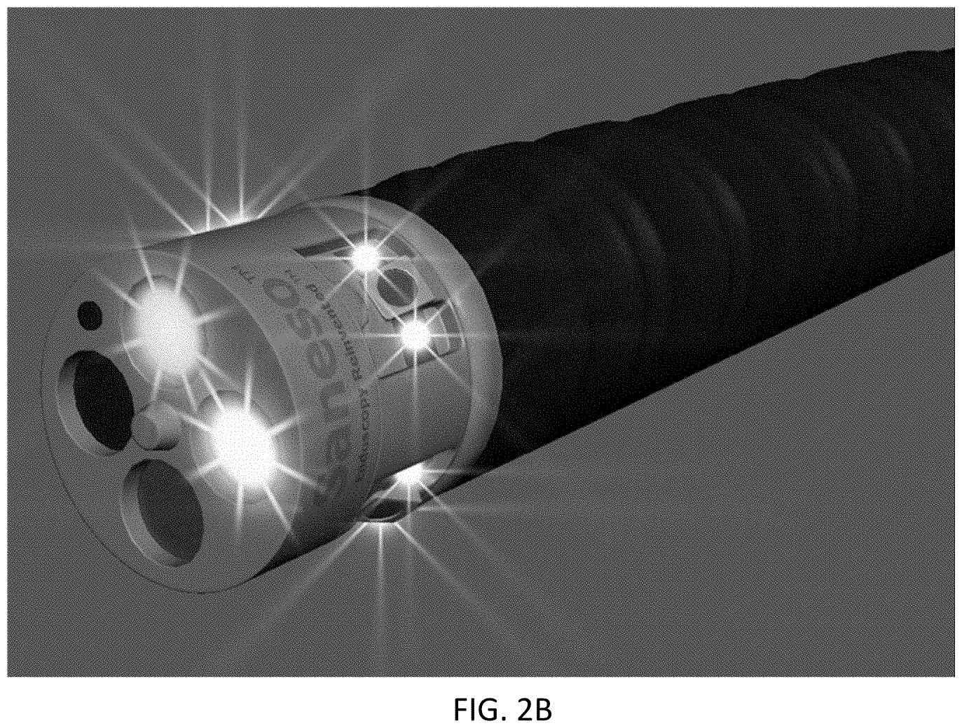

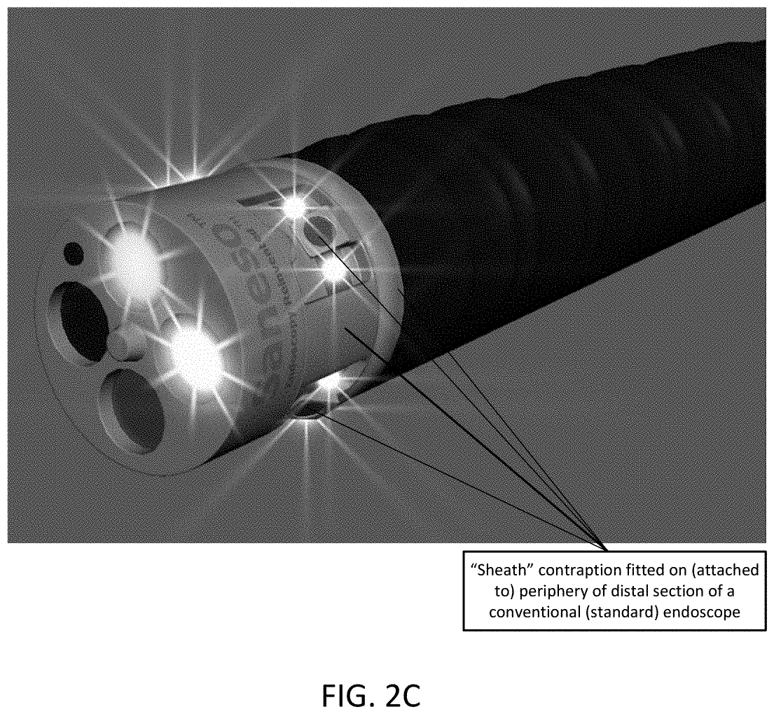

With reference now to the figures, and beginning with FIGS. 2A-E, FIGS. 2A-E illustrate a novel 360-degree panoramic view as well as exemplary configurations of the disclosed endoscope adapted to realize same, according to one or more embodiments of the present disclosure. The novel 360-degree panoramic view is a panoramic combination view programmatically formed by strategically combining and integrating multiple views each captured by a camera strategically placed at the distal end of the endoscope shaft. Hereinafter, the distal end of the endoscope shaft may also be referred to as “the distal end of the endoscope” or simply “the distal end”. In this example, the 360-degree panoramic view is formed by combining and integrating five different strategic participating views (which have been adapted to become sub-views of the 360-degree panoramic view), with the center sub-view being a front FOV captured by the front camera (see the exemplary front-facing camera shown in FIG. 2B), the left and right sub-views being left and right FOVs respectively captured by a left-facing camera and a right-facing camera (see the exemplary left-facing and right-facing cameras illustrated in FIG. 2B), and the top and bottom sub-views being top and bottom FOVs respectively captured by a top-facing camera and a bottom-facing camera (see the exemplary top-facing camera shown in FIG. 2B, noting that the exemplary bottom-facing camera is not specifically shown in FIG. 2B but should be readily understood as being positioned on the side of a vertical plane (which is a plane vertical to the longitudinal axis of the endoscope shaft where the exemplary top-facing camera is disposed) opposite to the side where the exemplary top-facing camera is disposed or otherwise positioned. The exemplary top, right, bottom and left sub-views may be “stitched” together along the four illustrated superimposed dashed lines using image-stitching techniques known in the general field of image-processing.

As used herein, terms such as “top-facing”, “bottom-facing”, “left-facing” and “right-facing”, when used to identify a camera disposed on or adjacent to the distal end of the shaft of the disclosed endoscope, each refer to a respective direction which the lens of the respective camera generally outwardly faces (or otherwise points to) relative to the front-facing distal end of the shaft when the distal end is in its natural (or, in other words, untwisted) form. A skilled artisan appreciates that since the distal section (which includes the distal end) of the shaft may be made of flexible material and thus flexible, the distal section of the shaft may be twisted in different manners during an endoscopy session (due to, e.g., the very limited space inside a colon under the endoscopy session) such that, e.g., an illustrated left-facing camera no longer generally faces (points to) left relative to the front-facing twisted distal end of the shaft, but instead generally faces (points to), e.g., right, top or bottom relative to the front-facing twisted distal end of the shaft. In such a case of a twisted distal end of the endoscope, this camera will still be referred to as the left-facing camera despite that the camera now outwardly faces (points to) another direction left relative to the front-facing distal end of the shaft. This discussion about the illustrated left-facing camera also applies to any of the illustrated right-facing, top-facing and bottom-facing cameras.

Hereinafter, as used herein, the terms “top camera”, “bottom camera”, “left camera”, and “right camera”, may be used interchangeably with the terms “top-facing camera”, “bottom-facing camera”, “left-facing camera”, and “right-facing camera”, respectively. In addition, the term “side-facing camera” may refer to any camera disposed on the periphery of the endoscope shaft (other than and not including the front face of the distal end of the endoscope shaft) adjacent to the distal end of the disclosed endoscope for capturing images surrounding the periphery of the endoscope shaft. Thus, the term “side-facing camera” may refer to any of the afore-illustrated top-facing, bottom-facing, left-facing, and right-facing cameras.

Also, in the descriptions herein, relative terms such as “left,” “right,” “vertical,” “horizontal,” “upper,” “lower,” “top” and “bottom” as well as any derivatives thereof (e.g., “left camera”, “horizontally”, etc.), should be construed to refer to a logical orientation as then described or shown in one or more drawing figures under the then discussion (unless such construing may conflict with any of other clarifications and guidance provided herein in connection with uses of terms). These relative terms are for convenience of description and are not intended to convey any limitation with regard to a particular orientation or direction.

In addition, although FIGS. 2B-C (or other applicable figures in the present disclosure) illustrate an exemplary configuration where there are one front-facing camera and four side-facing cameras associated with and corresponding to an integrated (consolidated) combination panoramic view as illustrated and exemplified in FIG. 2A (or other applicable figures in the present disclosure), a skilled artisan readily appreciate that other configurations each involving one or more front-facing cameras and multiple side-facing cameras (which may be fewer or more than the illustrated four side-facing cameras) may also be employed to be associated with and corresponding to a respective integrated combination panoramic view similar to the one (or ones) illustrated in FIG. 2A (or other applicable figures in the present disclosure), so long as the participating sub-views (each captured by a participating front-facing or side-facing camera) of respective integrated combination view are integrated, combined, and/or otherwise configured accordingly to form or substantially form an integrated combination panoramic view of 360-degree or substantially 360-degree.

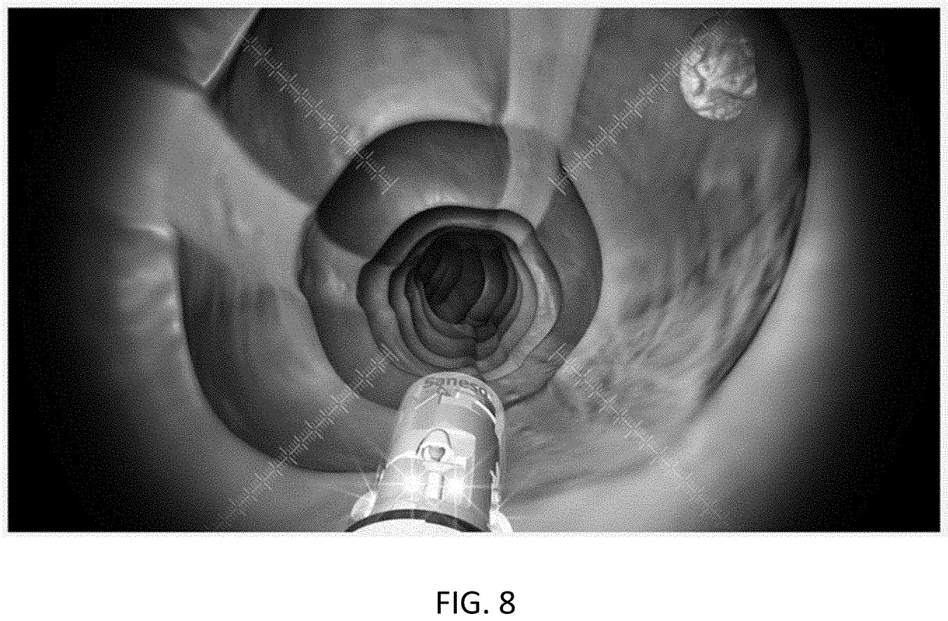

In one implementation, the circumference of the center circle (which sets the boundary of the center sub-view) drawn in solid line, and the set of four illustrated dashed lines emanating from the circumference of the center circle, are superimposed onto the 360-degree panoramic view to visually demarcate the set of five illustrated sub-views of the 360-degree panoramic view (namely, the center, left, right, top, and bottom, sub-views). In other implementations, the set of sub-views, or a subset of the set of sub-views, of the 360-degree panoramic view may be visually demarcated using one or more other visual demarcating schemes, without departing from the scope and the spirit of Applicant's disclosure. As one example, the set of illustrated dashed lines may be replaced by a corresponding set of dotted lines. As another example, a set of four measurement grids illustrated in FIG. 8 (which will be further described below) may be used to demarcate the top, left, bottom, and right sub-views (which is a subset of the set of five sub-views), with the boundary of center sub-view being transitioned into the top, left, bottom, and right sub-views.

FIG. 2C illustrates an embodiment of the disclosed endoscope where the illustrated left-facing right-facing, top-facing and bottom-facing cameras are disposed adjacent to and surrounding the distal end of the shaft of an otherwise conventional or standard endoscope through a sheath-like contraption (device) tightly (closely) fitted on, clipped onto, slipped over, or otherwise attached to, the outer periphery of a portion of the distal section of the shaft adjacent to the distal end of the shaft, such that the sheath-like contraption covers, or substantially covers, the outer periphery of the portion of the distal section of the shaft.

Aspects of this exemplary sheath-like contraption, such as its aspect of being closely fitted on and over the periphery of the illustrated portion of the distal section of the shaft, may be similar to aspects of the “sheath” device described in U.S. Patent Publication No. 2009/0023998 (which is hereby incorporated by reference in its entirety), except for, inter alia, the number of one or more anchored cameras, the respective locations of the respective one or more anchored cameras, and the respective directions which the respective one or more anchored cameras may face, with respect to each sheath-like device.

Specifically, as illustrated in FIG. 2C, the exemplary sheath-like contraption—besides being configured to be tightly fitted on, clipped onto, slipped over, or otherwise attached to, the outer periphery of the illustrated portion of the distal section of the shaft adjacent to the distal end of the shaft—may further be configured to securely anchor, or otherwise include, the left-facing, right-facing, top-facing and bottom-facing cameras at their respective designated locations on the exemplary sheath-like contraption, so that these cameras' respective left-facing right-facing, top-facing and bottom-facing configurations (with respect to the cameras' respective lenses) are realized. The exemplary sheath-like contraption may further be configured to securely anchor, or otherwise include, additional devices, such as LEDs illuminating for, or otherwise serving for, corresponding illustrated side-facing cameras (such as the exemplary left-facing, right-facing, top-facing and bottom-facing cameras).

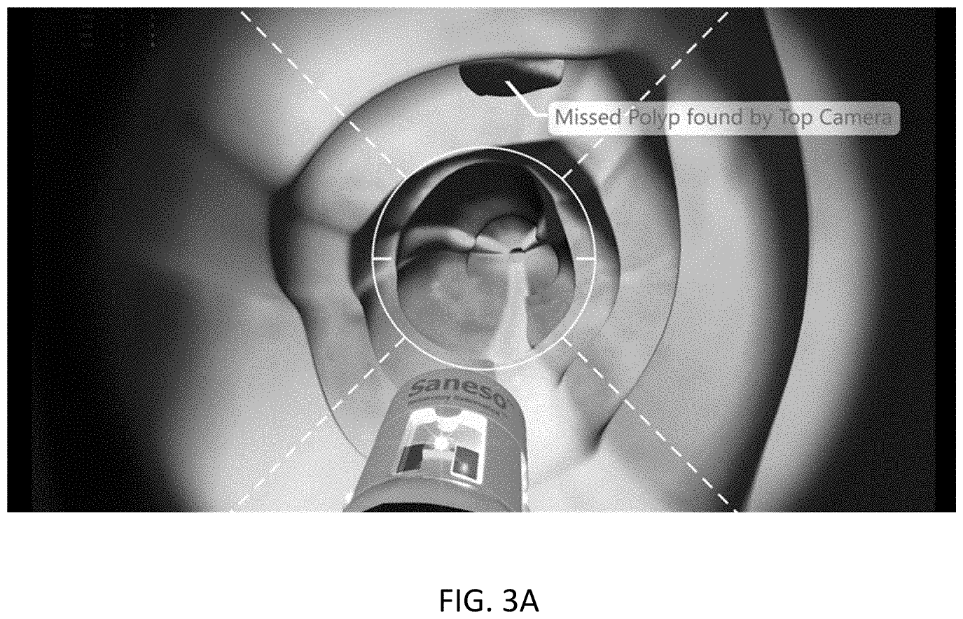

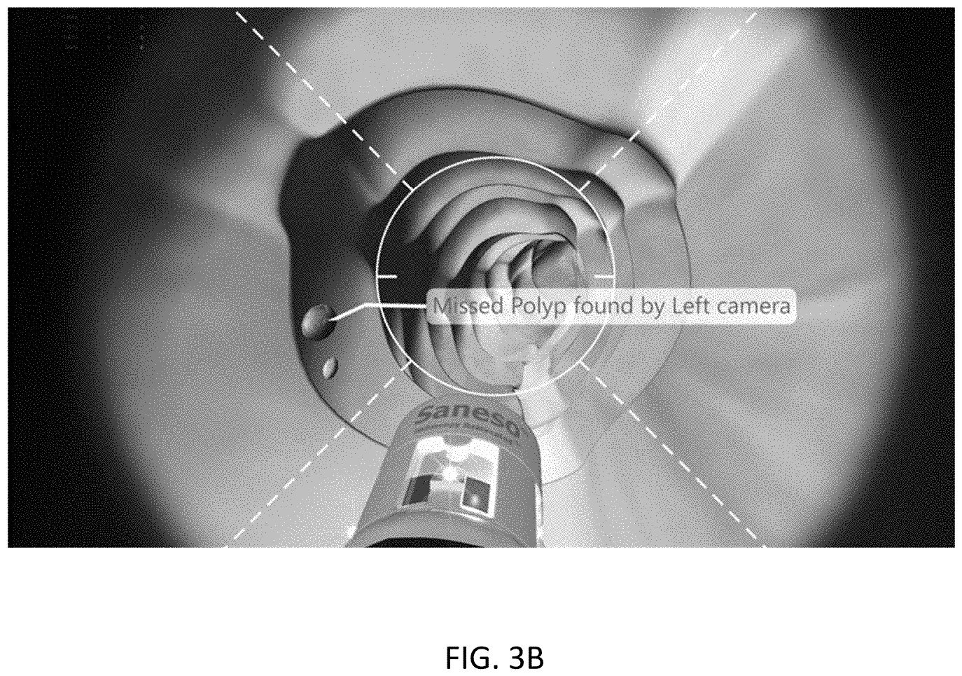

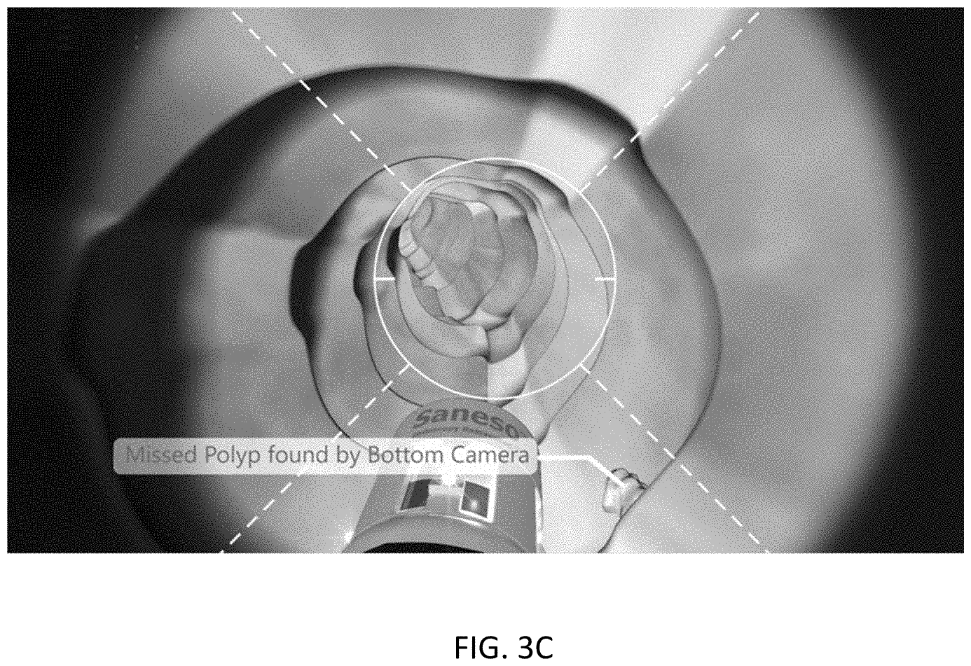

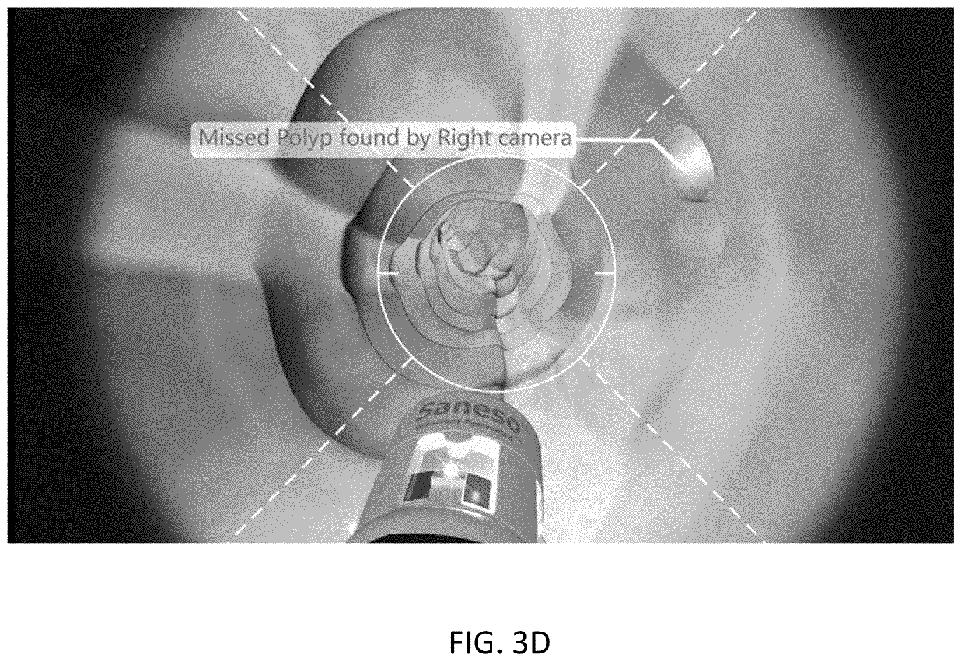

FIGS. 3A-D illustrate how the novel 360-degree panoramic view may be used to locate a polyp “hidden” from the center sub-view (a polyp which would have been missed by a conventional front-view-only endoscope, and may even likely be missed by a prior art multi-view endoscope not capable of forming, or otherwise not provided with, the novel 360-degree panoramic view), according to one or more embodiments of the present disclosure. As illustrated in FIG. 3A, as the endoscope is being withdrawn from deep inside a colon, the 360-degree panoramic view captures a polyp “hidden” behind one or more colon folds above the distal end of the endoscope—which may not be visible in the center sub-view (a front FOV) of the front camera during the withdrawal (due to, e.g., the visual obstruction by various different “awkward” spatial configurations of those colon folds above the distal end of the endoscope) and would have been otherwise easily missed even by a prior art multi-view endoscope nor capable of forming, or otherwise not provided with, the novel 360-degree panoramic view showing the top sub-view at all time during the withdrawal—via the top sub-view captured by the exemplary top-facing camera.

FIGS. 3B, 3C and 3D illustrate similar situations where, with the novel 360-degree panoramic view, a polyp “hidden” behind one or more colon folds at a location to the left of, below, or to the right of, the distal end of the endoscope—which may be not visible in the center sub-view of the front camera during the withdrawal and would have been otherwise easily missed even by a prior art multi-view endoscope not capable of forming, or otherwise not provided with, the novel 360-degree panoramic view showing a corresponding sub-view at all time during the withdrawal—via the left, bottom, or right sub-view captured by the exemplary left-facing, bottom-facing, or right-facing camera, respectively.

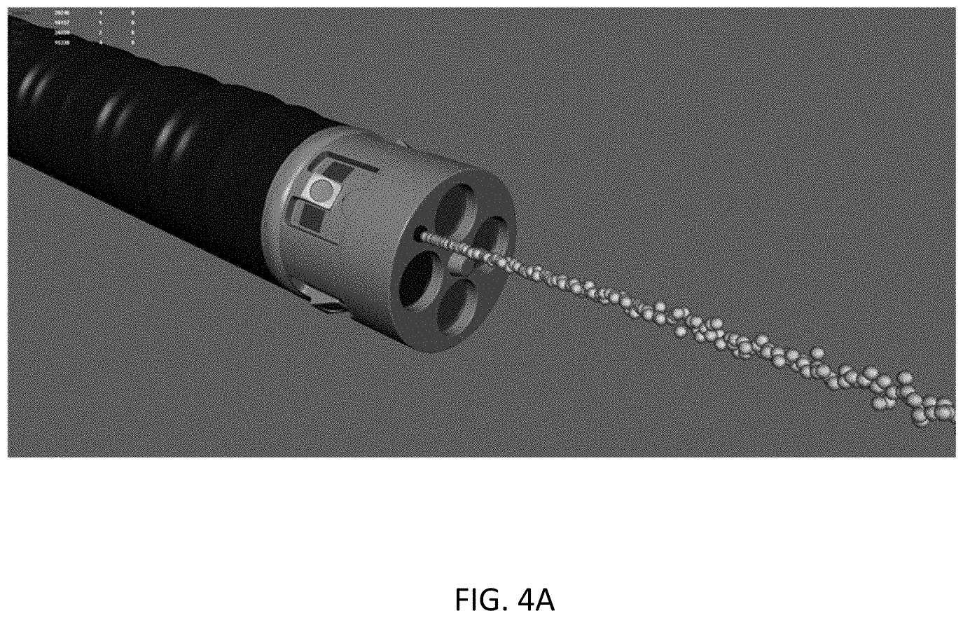

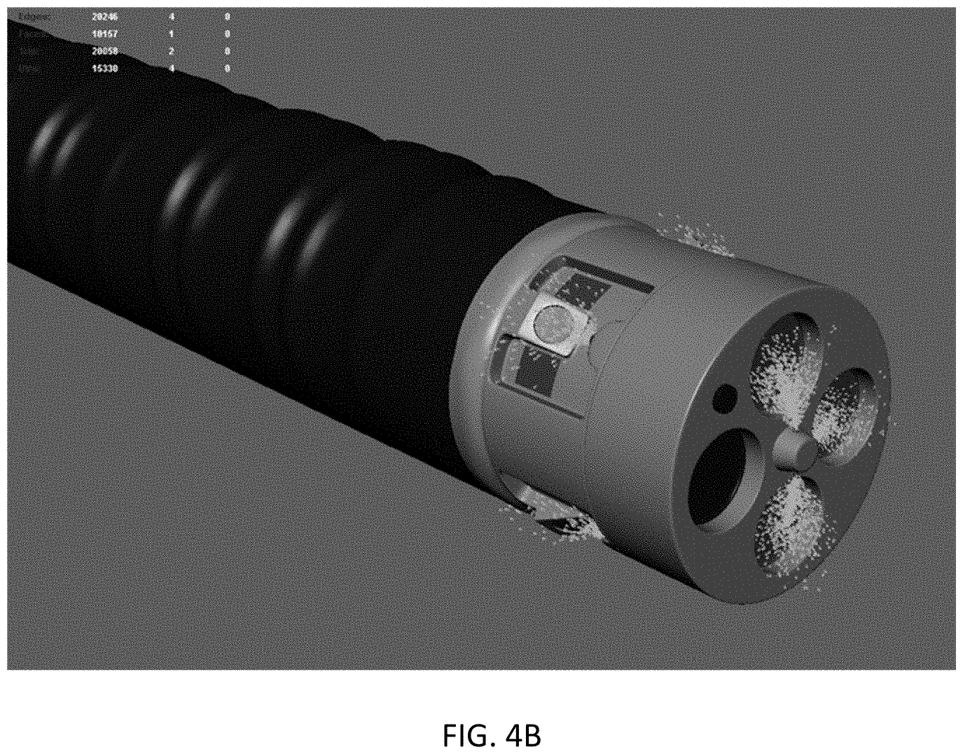

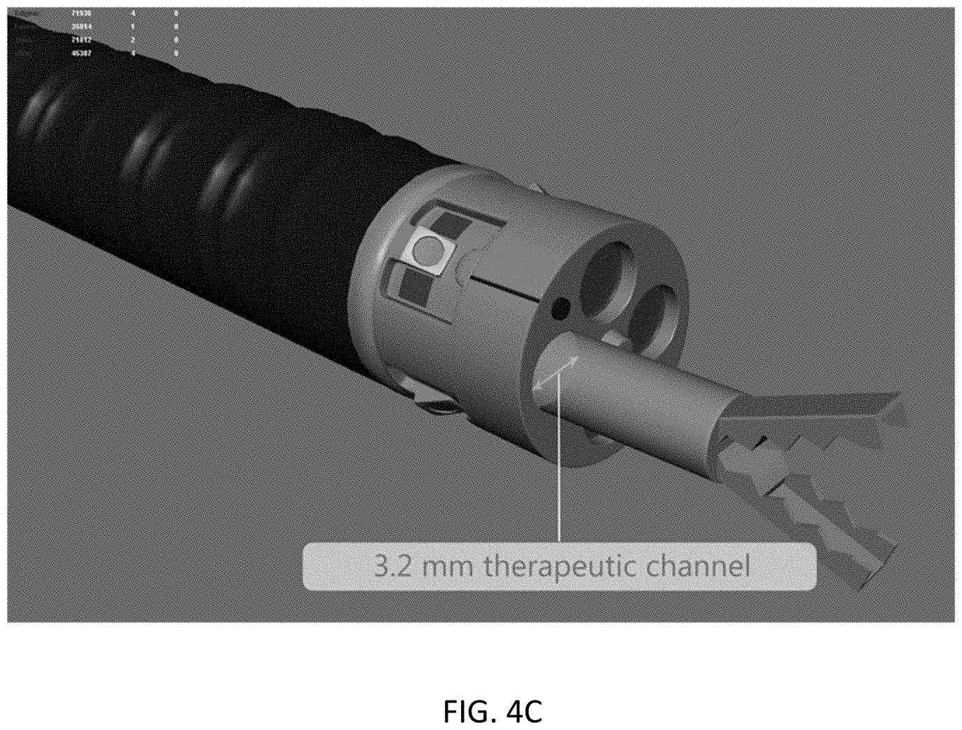

FIGS. 4A-C illustrate channels through which the disclosed endoscope (adapted to provide an embodiment of the novel 360-degree panoramic view) may be used to perform polyp (lesion) removal during a withdrawal of the disclosed endoscope from inside a colon, according to one or more embodiments of the present disclosure. FIG. 4A illustrates a front-facing main air or water channel on the front face of the distal end (close to the front view camera), which may be used to insufflate air or discharge a water jet/stream to, e.g., clean up things that are in front of the front face of the distal end of the endoscope (which may obscure viewing of what may be in front of the distal end). FIG. 4B illustrates accessory air or water channels located on different sides, or on/near the front face of, the endoscope shaft at the distal end. These are channels which may be used to channel and discharge air or water so as to, e.g., clean the shown top-facing, bottom-facing, left-facing, right-facing and front-facing cameras, respectively. FIG. 4C illustrates a front-facing main instrument channel on the front face of the distal end (close to the front view camera), which may be used to channel one or more suitable instruments to, e.g., remove a polyp located in an area (which may be referred to as a “therapeutic zone”) in the immediate outside front of the front face of the distal end of the endoscope shaft.

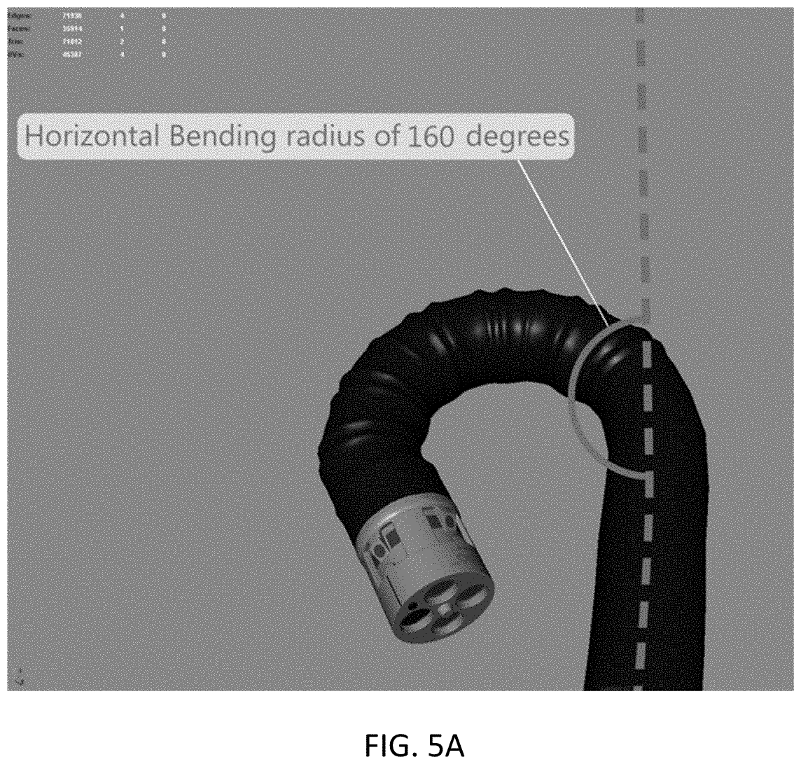

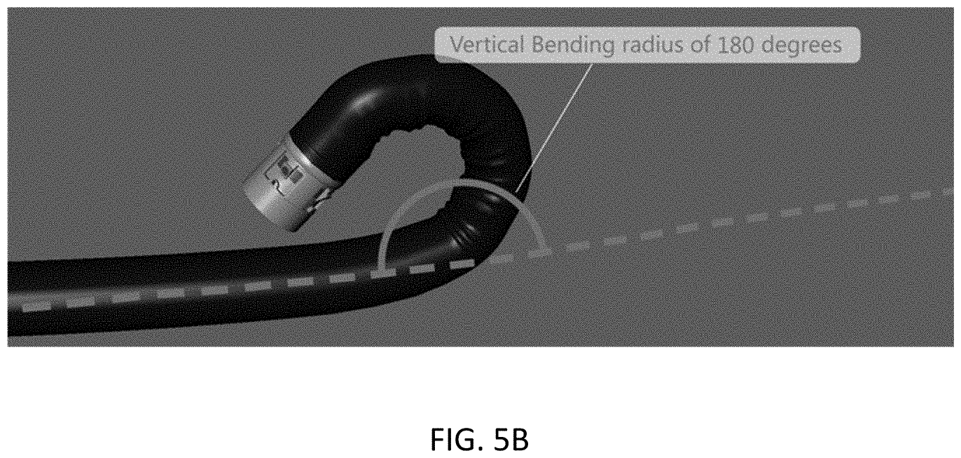

FIGS. 5A-B illustrate how the distal section of an embodiment of the disclosed endoscope may be flexed horizontally or vertically from a current location (position) to a new location (position), so as to advantageously change the direction which the front face of the distal end of the endoscope faces for the strategic purpose of, e.g., re-establishing a therapeutic zone in the immediate front of the front face of the distal end of the endoscope shaft, according to one or more embodiments of the present disclosure.

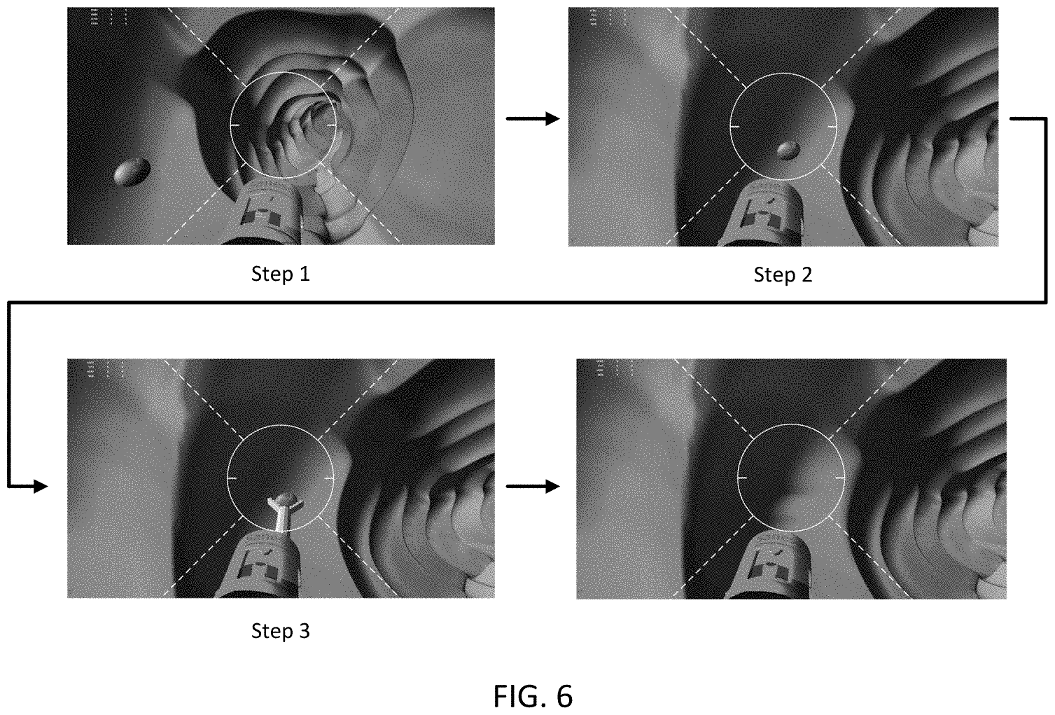

FIG. 6 illustrates an example of how an embodiment of the disclosed novel 360-degree panoramic view may work in concert (conjunction) with an embodiment of the disclosed endoscope (adapted to form and realize the 360-degree panoramic view) so as to effectively reduce polyp miss rate as well as target, and facilitate the removal of, a located and discovered polyp (lesion) during a withdrawal of the endoscope shaft from inside a colon, according to one or more embodiments of the present disclosure.

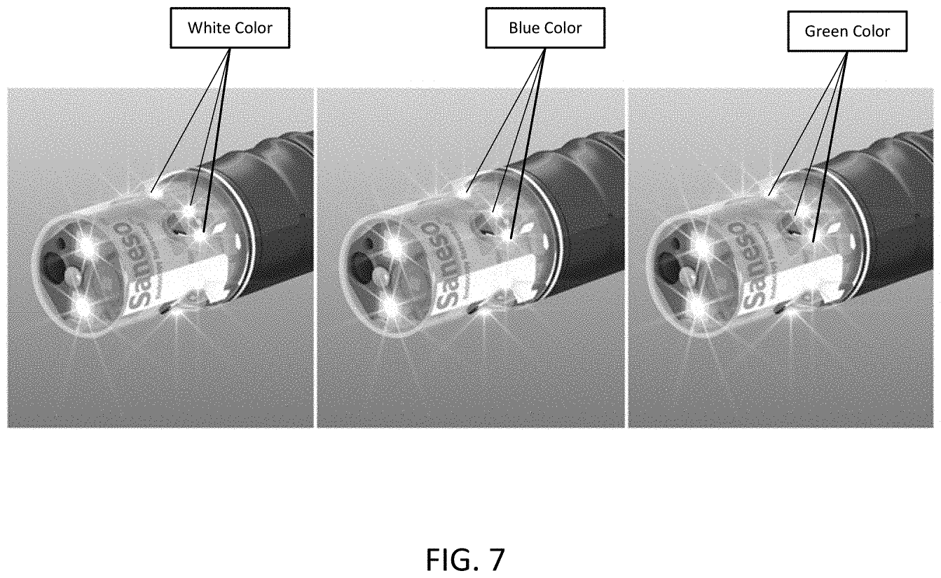

At step 1, the disclosed 360-degree panoramic view discovers and locates a polyp not visible in the center sub-view (a front FOV) of the front camera. If the polyp is shown inside the left sub-view—as visually demarcated from, e.g., the top and bottom sub-views through any suitable and effective demarcating means such as the one shown in FIGS. 2A and 6 or the one illustrated in FIG. 8 (as will be described below)—then an operator of the endoscope readily knows that images of the discovered polyp is captured by the left-facing camera. The left-facing camera, regardless of how the distal-end of the shift is twisted, may be readily located and identified through, e.g., the “Select Band” scheme illustrated in FIG. 7 and described below. Hence, in a first scenario where it is discovered or otherwise known that the distal end of the endoscope is in its natural untwisted form, the operator then knows that the polyp is located generally to the left of the front-facing distal end. In a second scenario where it is discovered or otherwise known that the distal end of the endoscope is twisted to such an extent that the left-facing camera now, e.g., faces generally right relative to the front-facing distal end, the operator then knows that the polyp is located generally to the right of the front-facing distal end (despite that the polyp is shown inside the left sub-view). In a third scenario where it is discovered or otherwise known that the distal end of the endoscope is twisted to such an extent that the left-facing camera now, e.g., generally faces upwards relative to the front-facing distal end, the operator then knows that the polyp is located generally above the front-facing distal end (despite that the polyp is shown inside the left sub-view). In a fourth scenario where it is discovered or otherwise known that the distal end of the endoscope is twisted to such an extent that the left-facing camera now, e.g., generally faces downwards relative to the front-facing distal end, the operator then knows that the polyp is located generally below the front-facing distal end (despite that the polyp is shown inside the left sub-view).

Similarly, if a lesion (polyp) is shown inside another sub-view (other than the left sub-view)—namely, any of the exemplary top, bottom or right sub-views—as readily appreciated by a skilled artisan, the general location of the polyp relative to the distal end of the endoscope may also be readily ascertained or otherwise known based on similar factors discussed above in connection with a polyp being discovered inside the left sub-view. As such, the novel 360-degree panoramic view facilitates, or otherwise enables, the operator to readily ascertain, or otherwise determine, the relative location information of a discovered polyp (namely, where a polyp discovered in any of its sub-views is currently generally located relative to the front-facing distal end).

At step 2, with the relative location information of the discovered otherwise “hidden” polyp—namely, information about where the current general location of the discovered polyp is relative to the front-facing distal end of the endoscope—readily obtained at step 1, the distal section of the disclosed endoscope is, inter alia, flexed or otherwise moved to have the distal end of the endoscope, and thus the front-face of the distal end, strategically repositioned, so that the discovered otherwise “hidden” polyp is now appeared within the center sub-view (a front FOV of the front-facing camera) and therefore is no longer “hidden” from the front FOV (captured by the front-facing camera) any more. That is, after the strategic repositioning of the distal end of the endoscope (and thus the front-face of the distal end), the polyp is now inside the re-established “therapeutic zone” in the immediate front of, and facing, the front face of the distal end of the endoscope (where the front-facing camera is disposed), a setup (configuration) which greatly facilitates removal of the discovered polyp during the withdrawal of the disclosed endoscope.

At step 3, with the distal end of the disclosed endoscope being strategically repositioned at step 2 and with the advantageous aid of the 360-degree panoramic view now showing the discovered polyp being inside the center sub-view (the front FOV captured by the front-facing camera disposed on or near the distal end of the endoscope shaft), the main instrument channel of the endoscope is, as shown in FIG. 6, readily leveraged and used, to channel one or more suitable instruments to have the one or more instruments extend outward beyond the distal end of the endoscope shaft, target, reach, and remove the discovered polyp with relative ease. Following step 3, as illustrated in FIG. 6, the discovered otherwise almost-certainly-would-have-been-missed “hidden” polyp is shown removed as the operator resumes the withdrawing of the endoscope from inside the colon to locate and remove additional one or more polyps (which may be potentially “hidden” from the front FOV of the front-facing camera disposed on or adjacent to the distal end of the disclosed endoscope).

FIG. 7 illustrates an innovative and novel lighting scheme which may be incorporated into the disclosed endoscope to realize the novel 360-degree panoramic view, according to one or more embodiments of the present disclosure. This advanced lighting scheme may be referred to as “Select Band” scheme. Specifically, as exemplified in FIG. 7, multiple modern long-lasting RGB LEDs may be deployed or otherwise provided on or near the distal end of the endoscope, and each of the afore-illustrated front-facing camera and side-facing cameras (which may include the afore-illustrated top-facing, left-facing, right-facing, and bottom-facing cameras) may be paired with (usually immediately adjacent to) one or more such modern LEDs. Each illustrated LED may be individually programmable (controllable) for selective color illumination and/or selective illumination intensity using known LED-related control technologies. Thus, through known programming or otherwise known control technologies on each of, each set of different sets of (e.g. with respect to a paired front-facing or side-facing camera), or all of, the provided LEDs (provided on or near the distal end of the endoscope), different lighting configurations (such as different patterns of colors and/or intensities) may be created as needed.

In one exemplary configuration, all illustrated LEDs (provided on or near the distal end of the endoscope) may illuminate with a first same color (e.g., a blue color) at a given point in time. In another exemplary configuration, at another given point in time, all illustrated LEDs may be switched to a second same color (e.g., a green color) for illumination. See each of the three exemplary configurations illustrated in FIG. 7 where the three respective colors selected for illumination of all LEDs are three different colors.

In one exemplary configuration, the color with which the set of one or more LEDs paired with (or otherwise serving for and providing lighting for) the afore-illustrated front-facing camera illuminate, may be different from the color with which the set of one or more LEDs paired with one of the afore-illustrated side-facing cameras illuminate. In another exemplary configuration, for two different afore-illustrated side-facing cameras, the two respectively paired sets of LEDs (with each set having one or more LEDs) may illuminate with two respectively different colors. Any of these two exemplary configurations or similar configurations may be particularly advantageous to identifying a specific side-facing camera even when the front-facing distal end of the disclosed endoscope shaft is twisted to such an extent that each side-facing camera no longer faces its respective general direction when the front-facing distal end is in its natural untwisted form. For example, if the set of one or more LEDs paired with (usually immediately adjacent to) the left-facing camera is configured to illuminate with a blue color while the other four sets of LEDs (respectively paired with the other three side-facing cameras and the front-facing camera) illuminate with respective colors which are all distinct from the blue color, then at different points in time, regardless of how many different directions which the left-facing camera may possibly face due to the respective different extents to which the front-facing distal end may be twisted inside, e.g., a colon, the left-facing camera may always be readily identified and located using the blue color with which its paired (usually immediately adjacent to) set of one or more LEDs illuminate (by, e.g., identifying and locating the blue color's general location or otherwise vicinity relative to the distal end of the endoscope).

In still another exemplary configuration, for each of the afore-illustrated side-facing cameras, the set of one or more LEDs paired with the respective side-facing camera may each illuminate with one or more different colors, respectively.

As readily appreciated by a skilled artisan, through known programming or otherwise known control technologies, the above-listed exemplary configurations of LED lighting, which are provided with respect to different light colors, may also be alternately or additionally provided with respect to different light intensities. Accordingly, this advanced “Select Band” scheme, when used in the exemplary steps illustrated in FIG. 6, gives an operator of the disclosed endoscope the flexibility to examine, e.g., lesions or polyps, in operator-selectable light spectra and/or light intensities with respect to LEDs, thereby affording an operator of the disclosed endoscope the freedom to optimize lighting in the vicinity of the distal end of the endoscope on a case by case basis.

FIG. 8 illustrates an innovative and novel “heads up display” scheme which may be incorporated into the disclosed endoscope, according to one or more embodiments of the present disclosure. To a great extent, this “heads up display” scheme may be similar to that used in advanced fighter jets. As illustrated in FIG. 8, the exemplary 360-degree panoramic view is superimposed thereon a set of four measurement grids, such that the respective sub-views showing the respective areas under the respective FOVs (field of view) of the four illustrated side-facing cameras may be demarcated from one another by the set of four measurement grids. In particular, the illustrated set of measurement grids may be leveraged to, inter alia, measure the size of a lesion during the exemplary steps illustrated in FIG. 6, as the lesion (polyp) is being discovered (or zeroed-in) prior to the operator commences to remove the discovered lesion.

Thus, with the exemplary superimposed (overlaid) set of measurement grids being part of the disclosed 360-degree panoramic view to demarcate the four respective sub-views of the four respective side-facing cameras from one another, this “heads up display” scheme, when, e.g., used in the exemplary steps illustrated in FIG. 6, provides a high level of spatial awareness and greatly assists lesion localization, measurement and targeting for therapeutic interventions.

A skilled artisan readily appreciates that this “heads up display” scheme may be implemented in one or more ways different from the one illustrated in FIG. 8 without departing from the scope and spirit thereof. For example, each measurement grid (of the illustrated set of measurement grids) may be used to measure the size of a lesion without also being used to set respective boundaries of the illustrated sub-views of the respective side-facing cameras. More specifically, a set of four measurement grids may be superimposed (on the rest of the 360-degree panoramic view) such that for each measurement grid, a good portion of the respective measurement grid lies inside (away from) the boundary of a respective sub-view of a respective side-facing camera, with an additional set of dashed lines being used to demarcate the four respective sub-views of the four respective side-facing cameras from one another (which is similar to the demarcating scheme shown in FIG. 2A).

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure.

What is claimed is:

1. An endoscope system comprising:
an endoscope, the endoscope having at its distal end at least one front-facing camera and multiple side-facing cameras, each of the front-facing and side-facing cameras capturing a corresponding participating sub-view; and
an image display device adapted to receive the corresponding participating sub-views, and integrate and combine the corresponding participating sub-views to form a combination panoramic view covering or substantially covering a vicinity of 360-degree or substantially 360-degree surrounding the distal end.
